(19) United States
(12) Patent Application Publication
(10) Pub. No.: US 2005/0234261 A1
(43) Pub. Date: Oct. 20, 2005

(54) Process for preparing cinnamic acids and alkyl esters thereof

(75) Inventors: Joerg Wilken, Hannover (DE); Andreas Kanschik-Conradsen, Garbsen (DE); Suzanne Wassmann-Wilken, Hannover (DE)

Correspondence Address: Scott Jacobson, Esq.;Honeywell International Inc., Law Department, AB2, P.O. Box 2245, Morristown, NJ, 07962-2245, US

(73) Assignee: Honeywell International Inc.

(21) Appl. No.: 10/824,259

(22) Filed: Apr. 14, 2004

(51) Int. Cl. C07C 69/00

(52) U.S. Cl. 560/129

(57) ABSTRACT

A process for producing cinnamic acids and alkyl esters thereof, particularly, fluorinated cinnamic acids and alkyl esters thereof. The process comprises reacting the appropriate bromobenzene and acrylic acid ester in a palladium-catalyzed HECK reaction under JEFFREY conditions using a phase-transfer catalyst (PTC) and an organic base to produce the corresponding cinnamic acid ester, and then preferably hydrolyzing the resulting ester under appropriate basic conditions, e.g. in the presence of a hydroxide, and precipitating the corresponding cinnamic acid product.

### FIELD OF THE INVENTION

This invention relates to an improved process for the manufacture of cinnamic acid and alkyl esters thereof, more particularly to an improved process for the manufacture of fluorinated cinnamic acids and alkyl esters thereof, and even more particularly to an improved process for the manufacture of 3,4-difluorocinnamic acid and alkyl esters thereof, particularly butyl 3,4-difluorocinnamate.

### BACKGROUND TO THE INVENTION

Cinnamic acid and its esters have a wide variety of uses, particularly in the perfume industry. More recently, derivatives, particularly esters of cinnamic acids, and more particularly esters of fluorinated cinnamic acids, have been discovered to be important intermediates and key substructures in various pharmaceutical drugs. Various patent and published patent applications illustrate the use of 3,4-difluorocinnamic acid as intermediates for adhesion cell inhibitors and alpha 1a andrenoceptor antagonists.

The patent publications and other published articles disclose various methods for the synthesis of various cinnamic acids and esters thereof. For example, US Patent Nos. U.S. Pat. No. 6,306,840 B1 and U.S. Pat. No. 6,376,538 B1 of Biogen, Inc. disclose the preparation of 3,4-difluorocinnamic acid tert-butyl ester and 4-fluorocinnamic acid tert-butyl ester via a reaction between an appropriate aldehyde (3,4-difluorobenzaldehyde and 4-fluoro-benzaldehyde) and the toxic and hazardous tert-butoxycarbonyl methylene triphenyl-phosphorane reagent resulting in yields of 88% and 91%, respectively. Steven D. Bull et al., Journal of the Chemical Society, Perkin Transactions, 2001, 23, 3112-3121, discloses that 3,4-difluorocinnamic acid butyl ester is prepared by a HORNER-EMMONS reaction using hazardous butyllithium as base. The yield of the butyl cinnamate given in this paper is 88%. PCT patent publications WO 2001092263 A1 and WO 2001092200 A1 of Astra Zeneca AB discloses and claims a synthesis of 3,4-difluorocinnamic acid (3-(3,4-difluorophenyl)-2-propenoic acid) via a KNOEVENAGEL condensation starting from the expensive 3,4-difluorobenzaldehyde and malonic acid with toxic pyridine and piperidine as bases. The reaction time is reported to be 4.5 hours and results in a yield of 88%. PCT Patent publications WO 2000027817 A1 and WO 2000027827 A1 of Merck & Co, Inc. describe the esterification of 3,4-difluorocinnamic acid with methanol in the presence of an acid to form the corresponding methyl cinnamate. Reaction details, the yield and the preparation of the starting material, 3,4-Difluorocinnamic acid, are not given. A. Ya. Aizikovich et al., Russian Journal of Organic Chemistry 1997, 33, 563-564: discloses that fluorinated cinnamic acids with various substitution patterns were prepared by coupling of fluorinated aryl bromides with acrylic acid in the presence of PdCl₂(PPh₃)₂ in DMF containing K₂CO₃. The disadvantages of this process include the fact that the expensive PdCl₂(PPh₃)₂ catalyst is moisture and oxygen sensitive and quantities of 10 mol % are required, and the yields are only ranging from 10-83%.

Other prior art methods for synthesis of fluorinated cinnamic acid esters include the following. Steven V. Ley et al., Chem. Commun. 2002, 10, 1134-1135, discloses a process using a polyurea-encapsulated Pd(OAc)₂ catalyst at a catalyst concentration of 2.5 mol % and a phase-transfer catalyst resulting in a yield of 75%. The reaction time for this process is not given. In the following described processes no phase transfer-agent is reported to be employed. M. Feuerstein et al., J. Org. Chem. 2001, 66, 5923-5925 and M. Feuerstein et al., Syn. Lett. 2001, 12, 1980-1982 disclose a process employing all-cis-1,2,3,4-tetrakis(Ph2PCH2)cyclopentane Pd complex catalyst at a catalyst concentration of 0.001 to 0.1 mol % producing a yield of 52 to 59% in a reaction time of 20 to 48 hours. A. C. Albeniz et al., J. Amer. Chem. Soc. 2001, 46, 11504-11505, discloses a process using a Pd complex, (NBu₄)₂[Pd₂(μ-Br)₂(C₆F₅)₂Br₂], as a catalyst producing a yield of 80% in a reaction time of 7 hours. M. R. Buchmeiser et al., J. Organomet. Chem. 2001, 1, 3946, discloses a process using a Pd complex catalyst, N-acetyl-N,N-bis(pyrimid-2-yl)amine palladium dichloride at an apparent catalyst concentration that seems to be <0.03 mol % producing a yield of 98% in a reaction time of 72 hours. L. Djakovitch et al., J. Organomet. Chem. 1999, 1, 16-26, discloses a process using [Pd(NH₃)₄](2+)-loaded NaY catalyst at a catalyst concentration of 0.1 to 1.0 mol % producing a yield of 72.6% in a reaction time of 20 hours. T. Dubuffet et al., Synth. Commun. 1999, 6, 929-936, discloses a process using tri-ortho-tolylphosphine/Pd(OAc)₂ catalyst at a catalyst concentration of 5.0 mol % in a reaction conducted over several hours, and no yield is reported.

There is, therefore, a need for an improved method of synthesis for preparation of cinnamic acids and esters thereof, particularly an improved method for synthesis of fluorinated cinnamic acids and esters thereof, and especially for the synthesis of 3,4-difluorocinnamic acid and esters thereof. A further need is to provide such improved method of synthesis that does not require the use of expensive aromatic aldehydes and that provides a more inexpensive method of synthesis than the traditional PERKIN or KNOEVENAGEL reactions based on such expensive aromatic aldehyde starting materials. A further need is to provide such a synthesis method that provides for ready recovery of expensive palladium catalyst. A further need is to provide such a synthesis method utilizing a highly active, i.e., high turnover number (TON), catalytic system that also enable easy recovery of expensive palladium catalyst. It is also desirable that such an improved method of synthesis be provided that produces the cinnamic acids and esters thereof in overall yields of about 90% or more, generally of about 95% or more. It would also be desirable for there to be provided such an improved synthesis process that uses a catalysis system that is highly active and selective at concentrations lower, and particularly, significantly lower than those proposed in the prior art.

### BRIEF DESCRIPTION OF THE INVENTION

Applicants have discovered an improved method of synthesizing cinnamic acids and esters thereof, particularly fluorinated cinnamic acids and esters thereof, and especially 3,4 difluorocinnamic acid and esters thereof employing the relatively cheap bromobenzene starting materials, particularly fluorinated bromobenzenes, and especially 1-bromo-3,4-difluorobenzene, and acrylic acid esters. The improved process comprises reacting the appropriate bromobenzene and acrylic acid ester in a palladium-catalyzed HECK reaction under JEFFREY conditions using a phase-transfer catalyst (PTC) and an organic base to produce the corresponding cinnamic acid ester. The ester can then be hydrolyed under appropriate basic conditions, e.g. in the presence of a hydroxide, and precipitating the corresponding cinnamic acid product. This process preferably produces the desired cinnamic acids and esters thereof in overall yields of about 90% or more, generally about 95% or more. The preferred process generally requires significantly reduced amounts of palladium catalyst, i.e., only about 0.01 mol %, compared to the amount required in prior art synthesis methods, i.e., 0.05 to 5.0 mol %. At the same time, a relatively small amount of PTC is generally required, i.e., only about 0.1 equivalents or less, whereas, in other reactions where a PTC is employed, usually 1.0 to 2.5 equivalents of PTC are required. Another feature of the preferred invention resides in the fact that the reaction does not require a large excess of acrylic ester starting material—generally only 1.0 to 1.05 equivalents of acrylate ester is sufficient to obtain optimum results. Another significant feature of the preferred invention is that stabilizing ligands such as the toxic and hazardous P(Ph)₃, phosphine, phosphate, carbene or thioether or oxygen and moisture sensitive Pd-complexes such as PdCl₂(PPh₃)₂ are not needed in the synthesis to obtain an efficient conversion of the starting materials. Additionally, the preferred reaction can be conducted in a polar, high boiling solvent, such as for example, N-methyl pyrrolidinone (NMP), dimethylformamide (DNF), dimethylacetamide (DMAA) and the like, and does not require additives such as N,N-dimethylglycine. Another preferred feature of the improved synthesis method of this invention is that the expensive palladium catalyst can be easily and readily recovered as Pd(0) particles, such as by filtration of the clear reaction mixture.

### DETAILED DESCRIPTION OF THE INVENTION AND PREFERRED EMBODIMENTS

The improved process of this invention comprises synthesizing cinnamic acids and esters thereof, particularly fluorinated cinnamic acids and esters thereof, and especially 3,4 difluorocinnamic acid and esters thereof employing the relatively cheap bromobenzene starting materials, preferably fluorinated bromobenzenes, and especially 1-bromo-3,4-difluorobenzene, and acrylic acid esters. To form the eater the improved process comprises reacting, in a first step, the appropriate bromobenzene and acrylic acid ester in a palladium-catalyzed HECK reaction under JEFFREY conditions using a phase-transfer catalyst (PTC) and an organic base to produce the corresponding cinnamic acid ester. In a further aspect, the resulting ester can be hydrolyzed under appropriate basic conditions, e.g. in the presence of a hydroxide, and precipitating the corresponding cinnamic acid product. This process produces the desired cinnamic acids and esters thereof in overall preferable yields of about 90% or more, more preferably about 95% or more, and generally requires significantly reduced amounts of palladium catalyst, i.e., only about 0.01 mol %, compared to the amount required in prior art synthesis methods, i.e., 0.05 to 5.0 mol %. At the same time, preferably, a relatively small amount of PTC is required, i.e., only about 0.1 equivalents or less, whereas, in other reactions where a PTC is employed, usually 1.0 to 2.5 equivalents of PTC are required. Another preferred feature of the improved synthesis method of this invention resides in the fact that the reaction does not require a large excess of acrylic ester starting material, generally only 1.0 to 1.05 equivalents of acrylate ester is sufficient to obtain optimum results. Another preferred significant feature of the improved method of this invention is that stabilizing ligands such as the toxic and hazardous P(Ph)₃, phosphine, phosphate, carbene or thioether or oxygen and moisture sensitive Pd-complexes such as PdCl₂(PPh₃)₂ are not needed in the synthesis to obtain an efficient conversion of the starting materials. Additionally, the preferred reaction can be conducted in a polar, high boiling solvent, such as for example, N-methyl pyrrolidinone (NMP), dimethylformamide (DNF), dimethylacetamide (DMM) and the like, and does not require additives such as N,N-dimethylglycine. Another preferred feature of the improved synthesis method of this invention is that the expensive palladium catalyst can be easily and readily recovered as Pd(0) particles, such as by filtration of the clear reaction mixture.

The preferred synthesis method is illustrated by the following reaction equations wherein the phase-transfer catalyst is indicated by PTC, n is an integer of from 0 to 5, preferably 1 to 5 and most preferably 2, and R is a straight, branched or cyclic alkyl group, preferably of from about 1 to 10 carbon atoms, and more preferably of from 3 to 8 carbon atoms, and most preferably of from 3 to 4 carbon atoms.

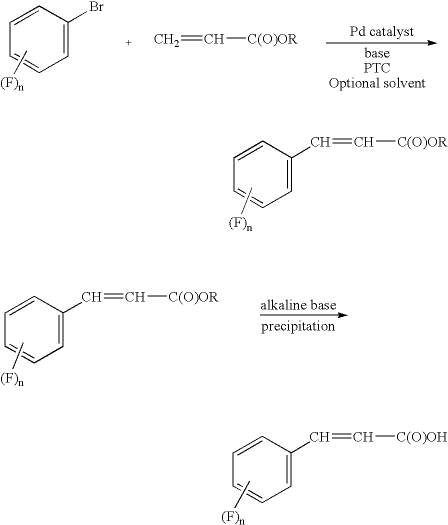

Preferably, the reaction equations are: 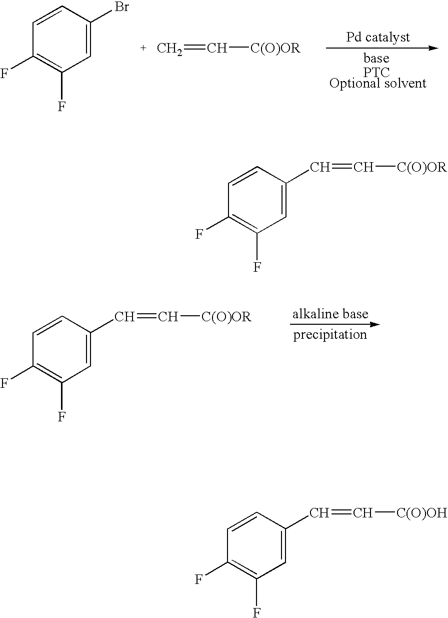
where R is alkyl of from 3 to 8 carbon atoms, preferably 4 carbon atoms.

The preferred palladium catalyst employed in the first reaction step of this invention may be any substantially phosphane-free palladium catalyst usable in Heck reactions, such as those disclosed in S. Braese and A. de Meijere in: “Handbook of Organopalladium Chemistry for Organic Synthesis, Ed. E.-i. Negishi, Wiley, New York, 2002, Vo. 1, pp. 1123-1368 and literature cited; S. Braese and A. de Meijere in “Metal-catalyzed Cross-coupling Reactions”, Ed. F. Diederich and P. J. Stang, Wiley-VCH, Weinheim, 1998, pp. 99-163 and literature cited therein; and T. Jeffery in “Advances in Metal-Organic Chemistry”, Ed. L. S. Liebeskind, JAI Press, Greenwich, Conn., 1996, Vol. 5, pp. 153-260, the disclosures of all of the above are incorporated herein by reference The palladium catalyst need not be phosphane-free. Another possibility for the palladium catalyst is palladium on carbon. Preferably, the palladium catalyst employed is Pd(OAc)₂, Pd(CI)₂, Pd(PPh₃)₄, (PdCl₂(PhCN)₂), and Pd(dba)₂ (palladium dibenzalacetone). A preferred palladium catalyst is palladium (II) acetate. The amount of palladium catalyst employed in the process of this invention can be a low as 0.008 mol % or less and up to about 5 mol %, and is preferably in an amount of from about 0.01 to about 5.0 mol %, more preferably in an amount of about 0.01 to about 0.02 mol %, per mole of bromobenzene reactant.

Any suitable phase-transfer catalyst (PTC) usable in Jeffrey conditions for a Heck reaction may be employed in the process of this invention, such as those mentioned in the articles cited in paragraph [0010] above and all are incorporated herein by reference. Examples of such suitable phase-transfer catalysts include but are not limited to tetraalkylammonium salts such as tetrabutylammonium hydrogen sulfate, trioctylmethylammonium chloride, tetrabutylammonium bromide and the like. The preferred PTC for the reaction scheme of this invention is tetrabutylammonium bromide. The amount of PTC employed in the process of this invention may be as low as about 0.05 equivalents or less and up to about 5 equivalents, preferably from about 0.1 to about 5.0 equivalent, more preferably up to about 1.0 equivalents, and most preferably about 0.1 equivalent, per mole of bromobenzene reactant.

The palladium catalyzed reaction with the PTC employed may utilize any suitable solid, liquid, or gaseous base. Examples of such suitable and preferred bases include, but are not limited to, liquid bases such as triethylamine, triisopropylamine, and tributylamine, and solid bases such as metal acetates, e.g., sodium acetate, and metal carbonates and metal hydrogen carbonates such as for example, sodium carbonate, potassium carbonate, and sodium hydrogen carbonate.

The alkyl acrylate reactant, CH₂═CH—C(O)OR, is preferably present in an amount of about 1.0 to 1.05 equivalents per mol of bromobenzene reactant, to reach optimum results. In a preferred embodiment a large excess of this reactant is not required.

The reaction may be conducted in any suitable polar high boiling solvent, preferably in one that has a boiling point of no greater than 120° C. Such polar, high boiling solvents include but are not limited to N-methylpyrrolidinone (NMP), dimethylformamide (DMF) and dimethylacetamide (DMAA). No additive such as N,N-dimethylglycine is required. The reaction is preferably conducted at a temperature of about 120° C. or more, preferably about 125° C. or more, and most preferably at a temperature within the range of about 130⁰° C. to about 140° C. Most preferably the reaction is conducted at a temperature of about 130° C. since temperatures above 130° C. generally do not significantly improve the outcome of the reaction with respect to yield or product purity. The reaction is generally complete in about 1 hour or less, and usually in about 30 minutes. Most preferably, the reaction of aryl bromide and alkyl acrylate is conducted in the presence of 0.02 mol % palladium catalyst, preferably palladium(II)acetate, 0.1 equivalent of the PTC, preferably tetrabutylammonium bromide (TBAB) and a base, preferably triethylamine (TEA), and at a temperature of about 130° C. for a period of about 30 minutes. The reaction generally results in an overall yield of cinnamate alkyl ester of about 95% or more. Under the reaction conditions described for this reaction step, quantitative yields of 98-100% (according to GC analysis) and >94% isolated yields of cinnamic acid alkyl esters are obtained.

In this reaction step of the invention the expensive palladium catalyst may be easily recovered as Pd(0) particles by a simple filtration of the clear reaction mixture.

The desired cinnamic acids, preferably the fluorinated cinnamic acids, and most preferably the 3,4 difluorocinnamic acid, are produced by hydrolysis, preferably by hydrolysis under basic conditions (e.g., aqueous alcoholic hydrolysis) of the cinnamate alkyl esters according to the reaction equation 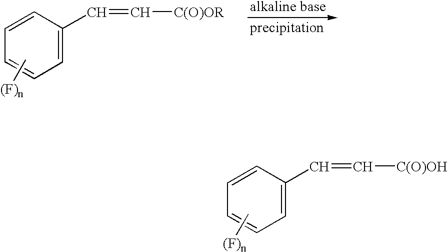
where R is an alkyl group having from 1 to 10 carbon atoms, preferably 3 to 8 and more preferably 3 to 4 carbon atoms, and n an is an integer of from 0 to 5, preferably 1 to 5 and more preferably 2 to 5, and most preferably 2. This step may use any suitable aqueous alcoholic base material, including but not limited to, aqueous NaOH, aqueous KOH and the like, preferably 2 m NaOH. Alternatively, an aqueous acid can be used to perform the hydrolysis or other hydrolysis techniques can be used as understood by one skilled in the art. Isolation (precipitation) of the cinnamic acid product, produces an isolated yield of a cinnamic acid in this hydrolysis step of generally about 95% or more with a GC purity of about 99% or more, generally at least about 99.58%. The isolated overall yield of both reaction steps in generally up to about 91%.

The invention is illustrated by the following representative, but non-limiting examples.

### EXAMPLE 1

### Production of Butyl 3,4-difluorocinnamate

A 1.0 L reactor, under N₂ atmosphere, was charged with 200 ml N-methyl-2-pyrrolidinone (NMP), 6.7 g tetrabutylammonium bromide (TBAB), 25.2 g triethylamine (TEA), 27.9 g butyl acrylate and 0.0093 g palladium(II) acetate and the reactor contents were heated to a temperature in the range of from 120° C. to 130° C. Then, with stirring, 40.0 g 1-bromo-3,4-difluorobenzene was added to the reaction mixture over a period of 30 to 60 minutes via an addition funnel. Stirring was continued for about 2 hours while the reaction temperature was maintained at 140° C. The reaction was complete after 30 minutes, with a GC assay of 94.4 area %. The reaction mixture was then permitted to cool down while stirring was continued. After cooling 100 ml water was added and a slight exotherm was observed with the temperature rising from 22° C. to about 30° C. The phases were then separated; the top aqueous layer was extracted with toluene (2×150 ml). The organic solutions were combined and washed with 100 ml 1M aq. HCl solution and then the organic layer was washed twice with water (2×50 ml) and then the phases separated. The organic solution was concentrated under reduced pressure in a rotary evaporator as the bath temperature was increased to 60° C. Crude yield: 49.9 g GC assay; 96.1 area %. Theoretical yield: 96.3%. Fractional distillation at 5 mbar using a 10 cm Vigurex column operated at a bath temperature of 171°-205° C.; sump temperature 152°-190° C.; head temperature 122°-125° C., resulted in a main fraction of 47.0 g, GC assay 98.4 area %. Yield 94.4%. The sump contained 2.1 g with 0.6 g other fractions.

### EXAMPLE 2

### Production of 3,4-Difluorocinnamic Acid

A 0.5 L reactor was charged with 92 ml 2M aqueous NaOH, 10 ml ethanol and 20.0 g butyl-3,4-difluorocinnamate produced in Example 1. The reaction mixture reached a pH of about 13.4. The reactor contents were heated to 80° C. and kept at this reaction temperature for about 2 hours. Conversion of the starting material butyl-3,4-difluorocinnamate was checked by TLC. The reactor was permitted to cool to room temperature. Another reactor was charged with 55 g 2M sulfuric acid and heated to about 50° C. to 60° C. The contents of the 0.5 L reactor were then added to this other reactor. Precipitation of the desired 3,4-difluorocinnamic acid occurs as the reactor contents are permitted to reach a pH of 2. The reactor contents was permitted to cool to room temperature and the contents then filtered in a 250 ml Büchner Trichner filtration apparatus. The solids were washed with 100 ml water and then dried in a rotary evaporator at 20 mbar with a bath temperature of up to about 65° C. Dried product obtained was 14.7 g, GC assay: 99.8 area %. Yield: 96%.

### EXAMPLE 3

### Preparation of Butyl 4-Fluorocinnamate and 4-Fluorocinnamic Acid

Employing 1-bromo-4-fluorobenzene reactant in place of 1-bromo-3,4-difluorobenzene reactant in Example 1, butyl 4-fluorocinnamate was produced, and employing this product in place of butyl 3,4-difluorocinnamte in Example 2, 4-fluorocinnamic acid was prepared. GC assay: 99 area %. The isolated yield of the ester is 93%

### EXAMPLE 4

### Preparation of Butyl Cinnamate and Cinnamic Acid

Employing bromobenzene reactant in place of the 1-bromo-3,4-difluorobenzene reactant in Example 1 and employing 0.1 mol % palladium catalyst, 0.1 equivalent PTA and a reaction temperature of about 140° C., butyl cinnamate was produced, GC results about 7% unreacted bromobenzene, isolated yield of butyl cinnamate was about 85%. Employing this product in place of butyl 3,4-difluorocinnamte in Example 2, cinnamic acid is prepared.

This invention provides a new synthesis process for cinnamic acid and alkyl esters thereof, particularly fluorinated cinnamic acids and alkyl esters thereof, with significantly higher yields than obtained in prior art processes, e.g., isolated yields of alkyl esters of >94% and isolated overall yield of cinnamic acids of generally 91% or more. These increased yields are obtained with significant reduction in terms of reagent quantities and costs. No great excess of phase-transfer catalyst (PTC) is required, generally only 0.1 to 1 equivalent need be employed. Similarly, the amount of palladium catalyst employed need only be about 0.02 mol %, generally about 0.01 to 0.02 mol % and does not require complex, expensive palladium structures. Further, no great excess of alkyl acrylate reagent need be employed, with only 1.0 to 1.05 equivalent being sufficient. Additionally, the synthesis process of this invention does not require stabilizing ligands such as P(Ph)₃, toxic and hazardous phosphine, phosphite, carbene, thioether or oxygen and moisture sensitive Pd complexes like PdCl₂(PPh₃)₂ to reach an efficient conversion of the starting materials. Also, no additive like N,N,-dimethylglycime need be used in the process.

While the invention has been described herein with reference to the specific embodiments thereof, it will be appreciated that changes, modification and variations can be made without departing from the spirit and scope of the inventive concept disclosed herein. Accordingly, it is intended to embrace all such changes, modification and variations that fall with the spirit and scope of the appended claims.

1. A process for the production of a compound comprising:
reacting a bromobenzene reactant of the formula: 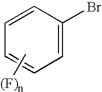
with an alkyl acrylate of the formula:
CH₂═CH—C(O)OR
in the presence of a palladium catalyst for a Heck reaction, a base, and a phase-transfer catalyst, to produce an alkyl cinnamate ester compound having the formula: 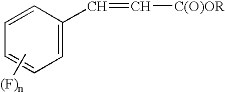
wherein n is an integer of from 0 to 5, and R is an alkyl group.

2. A process according to claim 1, wherein the palladium catalyst is a substantially phosphane-free palladium catalyst.

3. A process according to claim 1, wherein the reaction is conducted in the presence of an organic solvent.

4. A process according to claim 1, wherein n is an integer of from 1 to 5 and R is an alkyl group of from 3 to 8 carbon atoms.

5. A process according to claim 4, wherein n is an integer of 2 and R is a butyl group.

6. A process according to claim 1, wherein the palladium catalyst is employed in an amount of from about 0.008 to about 2 mol % per mol of bromobenzene reactant.

7. A process according to claim 6, wherein the palladium catalyst is employed in an amount of from about 0.01 to about 0.02 mol % per mol of bromobenzene reactant.

8. A process according to claim 1 wherein the phase-transfer catalyst is employed in an amount of from about 0.05 to about 5.0 equivalents per mol of bromobenzene reactant.

9. A process according to claim 8, wherein the phase-transfer catalyst is employed in an amount of from about 0.1 to about 1.0 equivalent per mol of bromobenzene reactant.

10. A process according to claim 9, wherein the phase-transfer catalyst is employed in an amount of about 0.1 equivalent per mol of bromobenzene reactant.

11. A process according to claim 1, wherein the palladium catalyst is selected from the group consisting of Pd(OAc)₂, Pd(Cl)₂, Pd(PPh₃)₄, (PdCl₂(PhCN)₂), Pd(dba)₂, and Pd on carbon.

12. A process according to claim 11, wherein the phase-transfer catalyst is a tetraalkylammonium salt.

13. A process according to claim 2 wherein the palladium catalyst is selected from the group consisting of Pd(OAc)₂, Pd(Cl)₂, Pd(PPh₃)₄, (PdCl₂(PhCN)₂), Pd(dba)₂ and Pd on carbon.

14. A process according to claim 13, wherein the phase-transfer catalyst is a tetraalkylammonium salt, and the bromobenzene reactant is 1-bromo-3,4-difluorobenzene.

15. A process according to claim 1, wherein the bromobenzene reactant is 1-bromo-3,4-difluorobenzene, the alkyl acrylate reactant is butyl acrylate, the palladium catalyst is Pd(OAc)₂, the phase-transfer catalyst is tetrabutylammonium bromide, the base is triethylamine, the reaction is conducted at a temperature in the range of from about 130° C. to about 140° C., the palladium catalyst is employed in an amount of from about 0.01 mol % to about 0.5 mol % per mole of bromobenzene reactant; the phase-transfer catalyst is employed in an amount of from about 0.1 to about 1 equivalent per mole of bromobenzene reactant, and the butyl acrylate reactant is employed in an amount of from about 1.0 to about 1.05 equivalent per mole of bromobenzene reactant.

16. A process according to claim 1 wherein the yield of alkyl cinnamate ester is >94%.

17. A process according to claim 1, wherein the palladium catalyst is a phosphane-free palladium catalyst and the palladium catalyst is employed in an amount of from about 0.008 to about 5 moles per mole of bromobenzene reactant, and the phase-transfer catalyst is employed in an amount of from about 0.05 to about 5 equivalents per mole of bromobenzene reactant.

18. A process according to claim 17, wherein n is an integer of from 1 to 5 and R is an alkyl group of from 3 to 8 carbon atoms.

19. A process according to claim 18, wherein the reaction is conducted in the presence of an organic solvent.

20. A process according to claim 19, wherein the bromobenzene reactant is 1-bromo-3,4-difluorobenzene, the alkyl acrylate reactant is butyl acrylate, the palladium catalyst is Pd(OAc)₂, the phase-transfer catalyst is tetrabutylammonium bromide, the base is triethylamine, the reaction is conducted at a temperature in the range of from about 130° C. to about 140° C., the palladium catalyst is employed in an amount of from about 0.0051 mol % to about 0.03 mol % per mole of bromobenzene reactant; the phase-transfer catalyst is employed in an amount of from about 0.1 to about 1 equivalent per mole of bromobenzene reactant, and the butyl acrylate reactant is employed in an amount of from 1.0 to about 1.10 equivalent per mole of bromobenzene reactant.

21. A process according to claim 20, wherein the yield of alkyl cinnamate ester is >94%.

22. A process according to claim 1, further comprising hydrolyzing the alkyl cinnamate compound under basic or acidic conditions to produce a cinnamic acid of the formula: 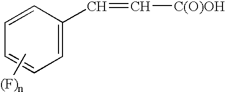
wherein n is an integer of from 0 to 5.

23. A process according to claim 2, further comprising hydrolyzing the alkyl cinnamate compound under basic or acidic conditions to produce a cinnamic acid of the formula: 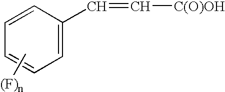
wherein n is an integer of from 0 to 5.

24. A process according to claim 3, further comprising hydrolyzing the alkyl cinnamate compound under basic or acidic conditions to produce a cinnamic acid of the formula: 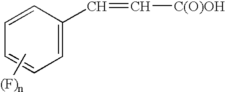
wherein n is an integer of from 0 to 5.

25. A process according to claim 4, further comprising hydrolyzing the alkyl cinnamate compound under basic or acidic conditions to produce a cinnamic acid of the formula: 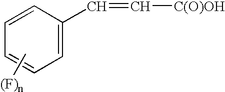
wherein n is an integer of from 1 to 5.

26. A process according to claim 6, further comprising hydrolyzing the alkyl cinnamate compound under basic or acidic conditions to produce a cinnamic acid of the formula: 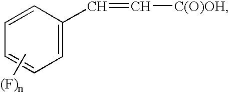
wherein n is an integer of from 0 to 5.

27. A process according to claim 8, further comprising hydrolyzing the alkyl cinnamate compound under basic or acidic conditions to produce a cinnamic acid of the formula: 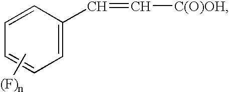
wherein n is an integer of from 0 to 5.

28. A process according to claim 11, further comprising hydrolyzing the alkyl cinnamate compound under basic or acidic conditions to produce a cinnamic acid of the formula: 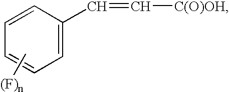
wherein n is an integer of from 0 to 5.

29. A process according to claim 15, further comprising hydrolyzing the alkyl cinnamate compound under basic or acidic conditions to produce a cinnamic acid of the formula: 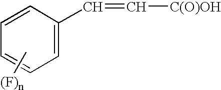
wherein n is the integer of 2.

30. A process according to claim 17, further comprising hydrolyzing the alkyl cinnamate compound under basic or acidic conditions to produce a cinnamic acid of the formula: 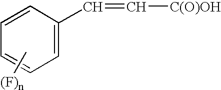
wherein n is an integer of from 0 to 5.

31. A process according to claim 20, further comprising hydrolyzing the alkyl cinnamate compound under basic or acidic conditions to produce a cinnamic acid of the formula: 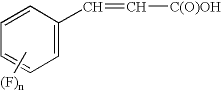
wherein n is the integer 2.

32. A process according to claim 31, wherein the yield of cinnamic acid is about 90% or more.
